(19) United States
(12) Patent Application Publication
(10) Pub. No.: US 2004/0024397 A1
(43) Pub. Date: Feb. 5, 2004

(54) Ablation catheter

(76) Inventors: Joseph C. Griffin III, Atco, NJ (US); David A. Jenkins, Flanders, NJ (US)

Correspondence Address: NORMAN E. LEHRER, P.C., 1205 NORTH KINGS HIGHWAY, CHERRY HILL, NJ, 08034, US

(21) Appl. No.: 10/393,490

(22) Filed: Mar. 20, 2003

Related U.S. Application Data
(63) Continuation of application No. 09/664,276, filed on Sep. 18, 2000, now abandoned.
(60) Provisional application No. 60/154,549, filed on Sep. 17, 1999.

(51) Int. Cl. A61B 18/14

(52) U.S. Cl. 606/41

(57) ABSTRACT

An ablation catheter designed to make long linear lesions by utilizing long flexible electrodes and conducting energy between the electrodes. One aspect of this invention includes the adding of a radiopaque material beneath the flexible electrodes. Another aspect is to co-extrude the conductor wires within the body of the catheter material. Another aspect of the invention is a junction box whereby defibrillating energy can be utilized through all electrodes on the catheter.

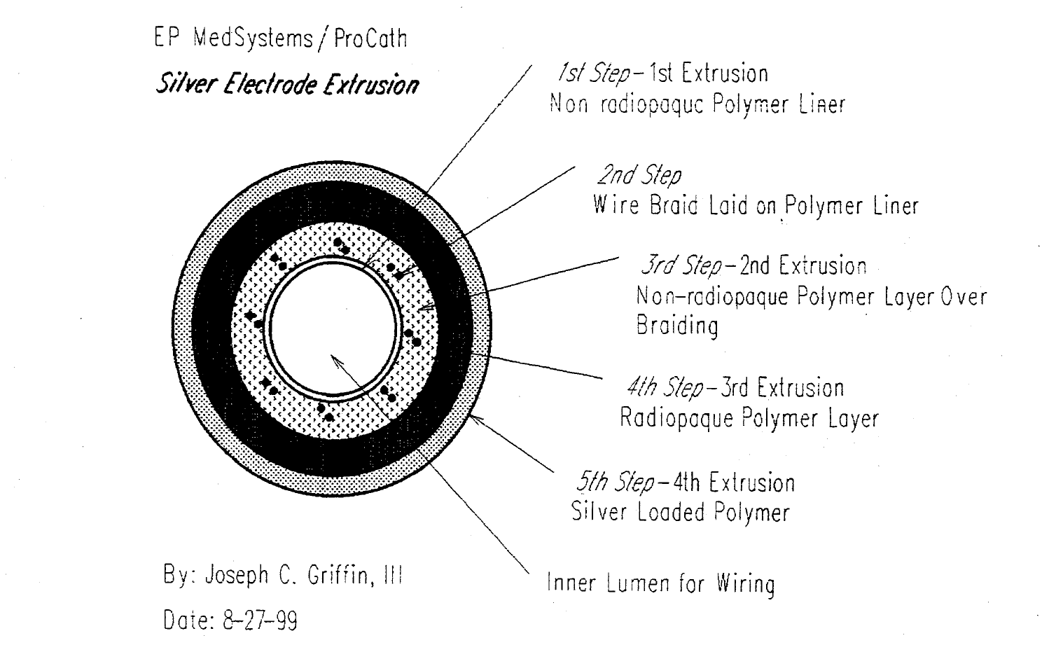

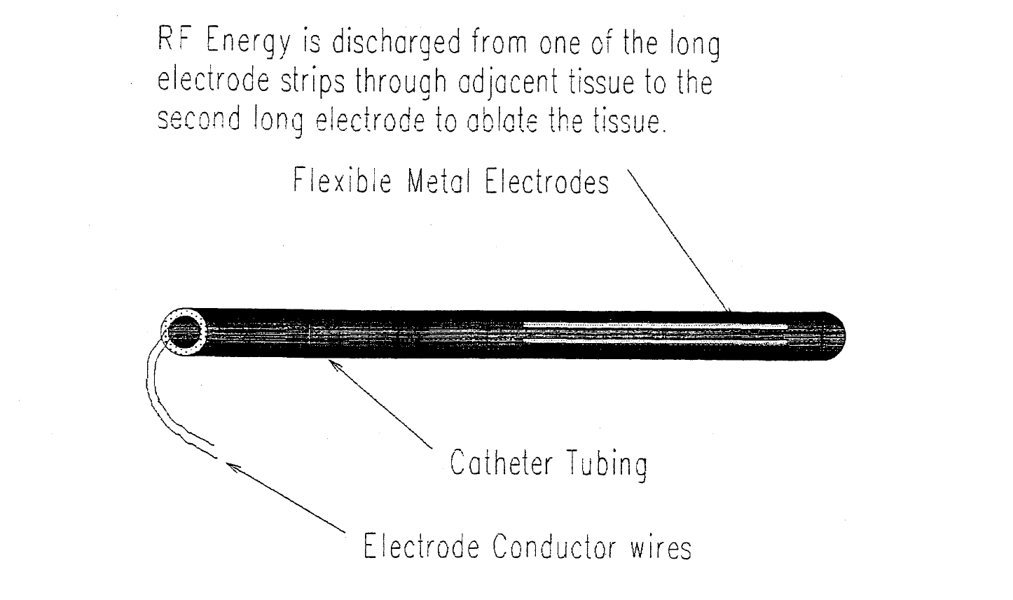

### BACKGROUND

Electrical Disorders of the Heart

Arrhythmias, which cause the heart to beat either too slowly or too rapidly, arise from numerous causes including heart tissue damage from previous heart attacks, congenital defects and certain diseases. Arrhythmias characterized by an abnormally slow heart rate (less than 50 beats per minute) are known as bradycardias, and are usually managed by an implanted pacemaker. Arrhythmias characterized by an abnormally fast heart rate (more than 100 beats per minute) are known as tachycardias or tachyarrhythmias. Tachycardia that originates in the ventricles is known as ventricular tachycardia (“VT”). The class of tachycardias that originate above the ventricles, including in the atria, are referred to as supraventricular tachycardias (“SVTs”). VT and SVTs can occur randomly on an occasional basis, or can be chronic and sustained, and both types of tachycardia can have life-threatening complications.

VT is a serious condition that can cause dizziness, unconsciousness and cardiac arrest. VT can also lead to ventricular fibrillation, in which the heart quivers and ceases to pump blood effectively. Without prompt medical attention, a patient suffering from ventricular fibrillation will die.

SVTs, while generally not fatal themselves, can cause serious and life-threatening complications. SVTs can be debilitating, causing chest palpitations, fatigue and dizziness. SVTs include atrial fibrillation, Wolff-Parkinson-White Syndrome, AV Nodal Reentry Tachycardia and atrial flutter. Atrial fibrillation is the most common and serious form of SW.

Current Diagnosis and Therapy of Tachycardia

Patients suspected of having tachycardia are initially screened by means of external cardiac monitoring. If tachycardia is confirmed or the initial screening is inconclusive, the patient may be referred to an electrophysiologist for a diagnostic procedure known as a cardiac EP study. During an EP study, specially designed electrode catheters (“EP catheters”) are percutaneously deployed and guided into the heart under X-ray fluoroscopy. The EP catheters then record electrical signals from inside the heart. These signals are transmitted to, displayed and stored on a computerized EP workstation. After the initial electrical signals from the heart have been examined, small amounts of electricity are delivered from an external stimulator through an EP catheter to the heart in order to stimulate a tachycardia. EP studies are undertaken to provoke tachycardias in a controlled setting, to locate the source of any electrical disturbance and to determine the nature of the arrhythmia. EP studies are also performed prior to the implantation of implantable cardiac defibrillators (“ICDs”) and to map the heart in conjunction with open chest procedures for the surgical treatment of arrhythmias.

Once the specific nature of an arrhythmia is identified, a therapeutic regimen is selected. Current therapies include drugs, surgery, ICDs, external cardioversion and therapeutic cardiac ablation. VT is treated primarily with drug therapy, ICDs and, in certain instances, therapeutic cardiac ablation. While antiarrhythmic drugs remain the most commonly employed treatment for VT, most of these drugs have undesirable side effects. Therapeutic cardiac ablation is a procedure in which a specialized EP catheter is used to burn a small lesion inside the heart to destroy abnormal conduction pathways, or tissue that cause arrhythmias. SVTs other than atrial fibrillation are primarily treated with therapeutic cardiac ablation. Because atrial fibrillation is characterized by random irregularity of electrical impulses in the atria that are extremely difficult to locate and destroy, therapeutic cardiac ablation as used today is generally ineffective as a treatment method for atrial fibrillation.

Atrial Fibrillation

Atrial fibrillation is a disorganized quivering of the upper chambers of the heart that results from aberrant conduction of electrical signals within the atria. This quivering leads to an ineffective and uncoordinated pumping of the heart, which often reduces cardiac output by up to 80% and causes impaired blood flow to the brain. In some patients, atrial fibrillation can lead to an uncontrolled ventricular heart rate, precipitating a life-threatening situation. Although patients with atrial fibrillation can be asymptomatic, most suffer from shortness of breath, palpitations, dizziness, fainting, or reduced tolerance to exercise and the activities of daily living. Atrial fibrillation is a significant cause of mortality and morbidity, particularly from thromboembolism and stroke. Each year approximately 75,000 strokes in the U.S. are related to atrial fibrillation

It is estimated that more than 2 million Americans are currently afflicted with atrial fibrillation and an estimated 160,000 new cases develop each year. Generally, atrial fibrillation is related to underlying cardiac disease, including congestive heart failure, coronary artery disease, hypertension and rheumatic heart disease, but it may also occur in patients with otherwise normal hearts. Atrial fibrillation is most commonly found in the elderly and it affects up to 5% of the population in the U.S. over the age of 60. It is the leading cause of arrhythmia-related hospitalizations and, in recent years, comprises the primary diagnosis for over 250,000 hospitalized patients, more than all other types of arrhythmia combined. Also, a large number of patients who were hospitalized for other reasons were found to have, or to have developed, atrial fibrillation during their hospitalization, further compromising their overall health.

The underlying causes of atrial fibrillation are complex and the progression of the disease varies from patient to patient. In patients with underlying cardiac disease, atrial fibrillation may initially occur paroxysmally, wherein short periods of atrial fibrillation are interspersed with normal heart rhythm. Paroxysmal atrial fibrillation generally converts back to normal heart rhythm spontaneously, but many patients require treatment with drugs and high energy external cardioversion to control their heart rate and associated symptoms. Although some patients may never progress beyond paroxysmal atrial fibrillation, the condition often precedes the development or chronic or persistent atrial fibrillation. In persistent atrial fibrillation, patients do not convert to normal heart rhythm spontaneously, but require cardioversion to terminate an episode and additional drug therapy thereafter to maintain normal heart rhythm. Persistent atrial fibrillation can progress to permanent atrial fibrillation, a condition in which the arrhythmia cannot be converted using traditional external cardioversion or drug therapy. Patients with permanent atrial fibrillation are generally given drugs to control the heart rate or therapeutic cardiac ablation is performed to destroy the AV node and a pacemaker is implanted to provide ventricular rate control. Neither treatment, however, addresses the underlying atrial fibrillation problem.

Although treatment of atrial fibrillation varies from patient to patient, the treatment goals remain constant: (1) restore and maintain normal heart rhythm, (2) control the ventricular heart rate and (3) prevent stroke. External cardioversion and drugs are used to restore normal heart rhythm; antiarrhythmic drugs are used to maintain normal heart rhythm; anticoagulants (blood thinning drugs) are used to reduce the risk of stroke; and drugs, AV node ablation accompanied by pacemaker implantation and open heart surgery are used to control the ventricular rate. None of these therapies is universally effective and each presents certain risks and side effects to the patient.

Restoration of normal heart rhythm, or cardioversion, is generally attempted by means of antiarrhythmic drug therapy or high-energy external cardioversion. A variety of drugs may be employed, but none are universally successful and antiarrhythmic agents generally have been shown to increase the risk of life-threatening ventricular arrhythmias. In addition, these drugs can have serious side effects, including liver failure, thyroid dysfunction, pulmonary fibrosis (thickening of the lungs), dizziness, nausea, difficulty urinating and diarrhea. Numerous studies report variable success with pharmacological cardioversion, with higher success rates reported in patients with recent onset atrial fibrillation of less than 48 hours duration.

High-energy external cardioversion is more effective than pharmacological cardioversion and is a mainstay of first-line therapy for atrial fibrillation. During external cardioversion, between one and four high energy electrical shocks of up to 360 joules each are applied across the chest wall by means of an external defibrillator. Because of the severe pain involved, patients undergoing external cardioversion are given general anesthesia or heavy sedation. This generally requires patient hospitalization and the presence of an anesthesiologist. In addition, patients experiencing atrial fibrillation for longer than 48 hours are routinely given anticoagulant drugs for two to three weeks before external cardioversion to reduce the risk of embolic strokes. Patients undergoing external cardioversion frequently report residual neuromuscular pain and experience skin burns. Serious side effects, while infrequent, include damage to heart tissue, spinal fracture, thrombus formation and stroke.

Patients who have undergone successful external cardioversion are frequently placed on a course of antiarrhythmic drug therapy to maintain normal heart rhythm. While external cardioversion is highly effective in terminating atrial fibrillation, without antiarrhythmic drug therapy, a majority of patients revert back to atrial fibrillation within one year of external cardioversion. With antiarrhythmic drug therapy, the percentage of patients reverting to atrial fibrillation decreases. Ninety percent of recurrences occur within the first six months. Despite these recurrence rates and the trauma and cost associated with high-energy external cardioversion, this treatment method remains a commonly employed first-line therapy and atrial fibrillation patients often require multiple external cardioversions.

One method of treating atrial fibrillation, or atrial flutter, is to mimic, via a catheter procedure, an open chest surgical procedure first pioneered by surgeon James Cox, whereby a number of long lesions are created in an effort to channel electrical conduction down corridors (or through a “maze”) created by the linear lesion. Cross talk, or horizontal conduction across the lesions is prevented, because the lesions, or scars, do not conduct the electrical activity.

A number of attempts to fabricate a catheter for use in ablation for atrial fibrillation or atrial flutter have been made, most utilizing hard metal electrode bands spaced apart to allow flexibility in the catheter, with RF energy applied to individual electrodes in alternating patterns, or applied across all electrodes at once while monitoring temperature periodically to prevent coagulum build up on the electrodes, or to prevent overheating and expulsion of tissue.

A desirable approach to create long linear lesions via a catheter is of course, to have a catheter with a long electrode. Using a hard metal band would make the catheter too stiff, so a number of inventions have been disclosed to overcome this problem. The state-of-the-art in the design of electrode catheters is quite advanced, and now appears to be a crowded field. Thus, this invention disclosure should be viewed as one of an evolution of the art, with distinct characteristics separating it from other thoughts existing in the prior art.

Willis, in U.S. Pat. No. 5,433,742, appears to have a fairly broad concept of thin conductive adhesive material for use as a long flexible electrode, and it appears that his concept would cover most any use of the electrode on a catheter. In Willis, pacing and recording through the electrode seems obvious, while one skilled in the art could add ablation and defibrillation as a use. No specific configuration of a combination of the Willis conductive adhesive bands was disclosed, and one objective of this patent is to provide a specific configuration of such conductive adhesive bands to aid in ablation, and specifically, ablation which creates long linear lesions.

Smeets, in U.S. Pat. No. 5,607,422, discloses a catheter using one single electrode, configured as a long metal strip down one side of a catheter, as a tool for making long lesions. The single strip would be in contact with the tissue (measured by the lowest impedance point) and ablation occurs by flowing energy through the single strip electrode conductor to a second conductor elsewhere in or out of the body, such as a ground patch under the patient's back. The electrode in contact with the tissue heats up the tissue and a lesion is formed. By putting the electrode down one side of the catheter, the energy is focussed into the tissue, and none is lost into the blood or other body fluids, thereby preventing the waste of energy and the creation of coagulum. However, Smeets does not contemplate using a second electrode on the same catheter for the conduction of the ablation energy.

Wang, in U.S. Pat. No. 5,462,545, seems to go one step further than Smeets, by designing three long strip electrodes around the catheter, each having slightly less than ⅓ of the circumference of the catheter tube and extending lengthwise, to be used in a coiled manner within the heart. Wang does not contemplate using only two electrodes, where one is the cathode and the other the anode to perfect much better control over the creation of the lesion. It appears that Wang's thrust is to use three or more electrodes to facilitate the optimal placement of one or more of the electrodes against the tissue, while selectively not activating the electrode(s) which do not contact the tissue.

In a co-pending application, Griffin creates a long electrode differentiated by a spiral cut through the length of the electrode, allowing the catheter to flex without damaging the electrode conduction. Again this is a single electrode, where the ground conductor is placed elsewhere within or outside the body.

This present invention teaches a method to create an ablation catheter with long flexible electrodes, where each pair of electrodes has both the anode and cathode for conducting the RF energy. The intervening space between the electrodes absorbs heat from the energy crossing between the electrodes and the tissue there becomes ablated. Unlike hard metal bands or strips, the flexible metal electrode material employed on this device has a very low thermal mass and therefore will adsorb very little of the resultant heat generated as RF power dissipates in the tissue adjacent to the paired-long linear electrodes. This is a desired characteristic of an ablation electrode to minimize coagulation in the surrounding blood pool.

A number of advantages are seen with this electrode configuration:

1. By focusing the energy, and having a short conduction pathway to the opposing electrode on the same catheter, less energy may be used. Less energy gives less chance of charring the tissue.

2. Correct placement of the catheter is assured by observing the time when the recorded signal from each electrode is equal, but opposite in nature, as the user will be recording differentially between the two electrodes.

3. With a given spacing between the electrodes, the amplitude and timing of the electrical energy waves can be altered so as to maintain control over the depth of the lesion. Lesion depth is critical in ablation of the atrium of the heart, as the wall of the atrium is relatively thin in comparison to other muscles of the heart.

Another object of this invention is to overcome the problem of viewing the electrode under X-ray fluoroscopy. Most electrode catheters have a limited amount of radiopaque material and the catheter shaft can be seen under the guidance of fluoroscopy. Normal electrodes, primarily the heavy metal bands, are quite dense and more radiopaque than the catheter shaft. Thus, each distinct electrode can be seen quite clearly under X-ray fluoroscopy. The electrode strips disclosed in Smeets and Wang could be of a relatively thick material, easily seen under X-ray. The thin material taught in Willis, however, gives poor visualization under X-ray, and cannot be used in electrophysiology or ablation procedures, where placement of the catheters is critical. Should this type of thin material be used to implement the Smeets or Wang electrodes, those catheters then become undesirable to use as the electrodes cannot be visualized.

A fairly standard version braided catheter tubing is now, under this present invention, co-extruded with a radiopaque polymer layer on its outer surface of a sufficient thickness to allow visualization under normal fluoroscopy during an EP procedure. The radiopaque layer of the extrusion is then removed in the isolation area that is required between each electrode. The result is a clearly defined beginning and ending edge of each electrode when viewed with fluoroscopy. After removal of the radiopaque material, the flexible metal electrode is sprayed on the catheter surface and processed in the same manner discussed in Griffin, U.S Pat. No. 5,888,577. Removal could be performed in a number of manners, but most likely an automated precise machine would be the best implementation. A laser machine could easily be adapted for this removal.

In manufacturing a catheter such as this, a tremendous amount of labor is used in running wires up the lumen(s) of the catheter, wrapping the wires around the catheter, and affixing the electrode material to the catheter. The catheter tubing has previously been extruded separately, with the conductor wires added by hand during the assembly process. It is yet another object of this invention to describe a method of manufacturing the catheter with the conductor wires extruded within the insulative tubing material. The wires can then be exposed to the outside electrodes by the use of a laser or similar automated finely tuned stripper machine. The advantage is a great reduction in labor time on the production of the catheter, and greater accuracy in the placement of the point where the conductor wire meets the intended location of the electrode.

The discussion herein has centered primarily around applications within the human heart, and specifically toward ablation of cardiac tissue. However, it is not intended that this invention be limited to cardiac applications. For example, and not by way of limitation, tissue ablation can be used as an attempted therapy within the pelvic region of the human body (for gynecological anomalies), within the throat (for snoring and sleep apneaanomalies), and within the lower esophagus (for esophageal sphincter and gastric reflux anomalies). Thus, it should be clear that the intent that this invention covers a number of applications which can be treated by a medical catheter or probe which may embody the overall theme(s) of this invention, and that the claims of this invention should be interpreted broadly to cover such other uses.

1. A catheter intended for use in the ablation of human tissue havingat least one pair of electrodes greater than 5 millimeters in length adhered to the catheter surface of the area toward the distal end of the catheter, said catheter containing isolated conductor wires connecting each electrode to an electrical connector at the proximal end of the catheter, where each of the electrodes within a pair are equal in size, length, and thickness and are positioned parallel to each other along the longitudinal axis of the catheter, separated by a certain defined spacing, where one electrode within a pair is considered the conducting anode, and the other electrode within the pair the conducting cathode, to be electrically conductive in order to create a linear lesion along the tissue interface essentially between the electrode pair.

2. The catheter in claim 1, where such electrode means is a flexible thin conductive adhesive material.

3. The catheter in claim 2, where the thin conductive adhesive material consists of silver, gold, platinum, or derivatives thereof (such as platinum-iridium), or a combination thereof.

4. The catheter in claim 3, where such material is applied to the catheter by an ion-beam deposition process, a sputtering process, or a spray-on type process.

5. The catheter in claim 1 has a conductive flexible electrode material co-extruded over a braided catheter shaft. A laser or similar mechanical means is used to etch electrode material away from the catheter surface effectively creating more than one electrode of varying sizes and configurations.

6. The catheter in claim 1, where such certain defined spacing between each electrode within a pair is essentially between 0.5 millimeter and 0.5 centimeter.

7. The catheter in claim 1, where each electrode within the pair extends around the circumferential axis no more than a length approximately one fourth to one third of the length of the total circumference of the catheter.

8. The catheter in claim 2, where the thickness of the thin conductive adhesive material is essentially between 0.1 microns and 50 microns.

9. A method of manufacturing an electrode catheter, with one or more conductor wires extending from an electrical connector on the proximal end through the catheter to the distal portion of the catheter, including the steps of manufacturing as follows:
1) Stripping the catheter surface away from the conductor wire(s) by laser means,
2) Filling in the “stripped” area with a conductive potting material so as to form an essentially smooth outer catheter diameter with a conductive area exposed, connected to the inner conductor wires.

10. The method of claim 9, also comprising the step of adhering a further electrode material to the outer housing of the catheter so as to encompass the area of the potted material.

11. The method of claim 10, where such electrode material is applied via ion beam deposition, sputtering, or spray.

12. The method of claim 10, where such electrode material is platinum, silver, gold, or a derivative thereof, or a combination thereof.

13. The method of claim 10, where such electrode material is a hard metal band.

14. The method of claim 9, further comprising the step of co-extruding the conductor wire as a partof the catheter tube body.

15. The method of claim 10, where such electrode material is a thin, flexible conductive material.

16. An electrode catheter, including an insulative catheter body comprised of an elongated flexible member having a distal end and a proximal end, one or more electrodes adhered to the area at the distal end, connected to the proximal end via conductor wires, where such conductor wires are imbedded within, and extruded as part of, the insulative catheter body, as opposed to being contained within a hollow portion or lumen of the catheter.

17. An electrode catheter, containing one or more thin flexible electrodes, where such electrodes contain a radiopaque lining between the electrode material and the main catheter shaft.

18. A catheter in claim 17, where the thin flexible electrodes are made from ion beam deposition, sputtering, or spray.

19. A catheter in claim 17, where the thin flexible electrodes are made of gold, platinum or silver, or a derivative thereof or a combination thereof.

20. A catheter in claim 17, where the radiopaque lining contains the material bismuth or barium or other suitable biocompatible radiopacifier.

21. A method of manufacturing a catheter, wherein a radiopaque outer layer of material is extruded as an integral part of the extruded tubing, and wherein such radiopaque outer layer is defined, or etched, by use of laser.
